(19) United States
(12) Patent Application Publication
(10) Pub. No.: US 2025/0099286 A1
(43) Pub. Date: Mar. 27, 2025

(54) OSTOMY POUCH ACCESSORY

(71) Applicants: LOHMANN GMBH & CO. KG, Neuwied (DE); COVESTRO DEUTSCHLAND AG, Leverkusen (DE); WITTE TECHNOLOGY GMBH, Munster (DE)

(72) Inventors: Volker LUTZ, Neuwied (DE); Gerd BÜSCHEL, Admannshagen-Bargeshagen (DE); Klas-Moritz KOSSEL, Verden (DE); Philip RENNERS, Munster (DE)

(21) Appl. No.: 18/832,336

(22) PCT Filed: Jan. 25, 2022
(86) PCT No.: PCT/EP2022/051609

§ 371 Date: Jul. 23, 2024

(51) Int. Cl. A61F 5/44

(52) U.S. Cl. CPC A61F 5/4404

(57) ABSTRACT

To increase a comfort and a flexibility, an ostomy pouch accessory for usage with at least one ostomy pouch device is proposed, comprising at least one sensor element for a detection of a leakage of the ostomy pouch device in at least one application state, also comprising at least one adhesive element for a detachable fixation of the ostomy pouch accessory in the application state.

### CROSS-REFERENCE TO RELATED APPLICATIONS

The present application claims priority to PCT patent application PCT/EP2022/051609, “Stomabeutel-Zubehör” (Ostomy pouch accessory), filed Jan. 25, 2022, which is hereby incorporated by reference.

### FIELD OF TECHNOLOGY

The present technology relates to an ostomy pouch accessory and a method of using an ostomy pouch accessory.

### BACKGROUND

It is known from the state of the art to equip ostomy pouch devices with integrated sensors configured to detect a leakage of the ostomy pouch device. However, the integration of these sensors does not allow for an easy way to upgrade ostomy pouch devices for which the sensors have not been integrated during the manufacturing process. Furthermore, there is no option to detach the sensors from the ostomy pouch device and to re-attach them, which prevents an easy replacement of the sensors, for example if they get damaged.

It is a preferred object of the present technology to surpass this disadvantage at least partially. Furthermore, it is an object of the present technology to provide a generic device with improved features relating to a function or design. This object is achieved by the features of claims 1 and 14, while other embodiments and improvements of the present technology may be taken from the dependent claims.

### BRIEF SUMMARY

The present technology relates to an ostomy pouch accessory for usage with at least one ostomy pouch device.

### BRIEF DESCRIPTION OF FIGURES

FIG. 1A schematic depiction of a system with an ostomy pouch device and an ostomy pouch accessory in a cross-sectional view.

FIG. 2A schematic depiction of the system in a top view.

FIG. 3A schematic depiction of the system in a closer top view.

FIG. 4A schematic chart for a method of using the ostomy pouch accessory.

It is proposed that the ostomy pouch accessory comprises at least one sensor element for a detection of a leakage of the ostomy pouch device in at least one application state and comprises at least one adhesive element for a detachable fixation of the ostomy pouch accessory in the application state.

This way, a design providing increased comfort and flexibility can be achieved. Advantageously, the user may choose when and how often to attach the ostomy pouch accessory to the ostomy pouch device. Especially advantageously, ostomy pouch devices which are already attached to a user can be equipped with the ostomy pouch accessory retroactively and without needing special tools or trained staff. For example, the ostomy pouch accessory may be attached to the ostomy pouch device before performing an activity relating to sports and removed after the activity. Furthermore, the ostomy pouch device and the ostomy pouch accessory may be sold separately, allowing users that would only rarely need the ostomy pouch accessory to avoid having to pay unnecessary additional costs.

Preferably, the ostomy pouch accessory may be attached to and detached from the ostomy pouch device any number of times. Advantageously, the ostomy pouch accessory may be attached to different, identical ostomy pouch devices, especially advantageously, the ostomy pouch accessory may be attached to different, non-identical ostomy pouch devices. Preferably, the ostomy pouch accessory can be adjusted to be used with ostomy pouch devices of different sizes. Especially preferably, the ostomy pouch accessory may be stretched and/or folded for usage with different ostomy pouch devices. Further preferred is that the ostomy pouch accessory is available in different sizes and/or shapes, each of which being configured to be used with a corresponding type of ostomy pouch device.

The ostomy pouch device preferably comprises at least one opening, which is surrounded by a hydrocolloid plate, the hydrocolloid plate being adhered to the skin of the user in an application state and surrounding a stoma of the user. The ostomy pouch device also comprises an ostomy pouch, which is connected to the hydrocolloid plate and configured to take in material exiting the stoma. The ostomy pouch device is preferably configured for long-time usage but may be detached and re-attached or swapped with a fresh ostomy pouch device in the case of, for example, a damaging of the ostomy pouch device. In case of a leakage, which may for example be caused by a blockage of the stoma, materials often exit between the hydrocolloid plate and the skin of the user, potentially leading to skin irritations, inflammation and embarrassment of the user as well as inconvenience caused by smells. In most commercially available ostomy pouch devices, the opening is circular.

The sensor element preferably comprises at least one trace, preferably at least two traces for a detection of the leakage. The trace may consist of copper and/or aluminum. Preferably, the sensor element detects the leakage by constantly measuring a local moisture. The sensor element may measure the local moisture via a capacitive measurement and/or a measurement of the electrical conductivity in a near vicinity of the trace. Preferably, the sensor element measures the local moisture within a sensor area, the sensor area being defined as an area of the substrate arranged between two traces. The at least one trace of the sensor element may have any shape, for example, the trace may have an open or closed shape. Furthermore, the trace may either be devoid of turns or comprise at least one turning point in which two parallel sections of the trace meet. Additionally, the trace may comprise at least one projection and/or at least one meandering section to improve a detection of the leakage. Advantageously, neighboring sensors are at least partially intertwined.

The application state is to be understood as a state in which the ostomy pouch device is adhered to the skin of the user and the ostomy pouch accessory is attached to the ostomy pouch device. Preferably, the application state is a state in which both the ostomy pouch device and the ostomy pouch accessory provide all of their intended functions.

Preferably, the adhesive element comprises at least one adhesive area comprising an adhesive, especially preferably an adhesive from a group consisting of thermoplastics or elastomers. Preferably, the adhesive area is configured to be usable with different types of ostomy pouch devices in a flexible manner. Preferably, depending on a size and/or shape of the ostomy pouch device, different sections of the adhesive area may be used to attach the ostomy pouch accessory to the ostomy pouch device. Preferably, the adhesive area forms the entire adhesive element. Alternatively, the adhesive element may comprise at least one further, non-adhesive area. The adhesive element preferably has a width of 1 to 50 cm, further preferably of 2 to 20 cm and especially preferably of 5 to 10 cm. The adhesive element preferably has a length of 2 to 100 cm, further preferably of 5 to 70 cm, especially preferably of 10 to 50 cm.

Preferably, the adhesive element has a breathability of at least 300 g/m²/24 h. This way, skin breathing can be enabled and skin damages may be prevented even during long-time usage of the ostomy pouch accessory. The adhesive area preferably comprises or consists of an adhesive selected from a group consisting of silicon adhesives, acrylate adhesives, polyurethane adhesives, rubber adhesives as well as combinations of at least two of these and/or further adhesives known from the field of medical plasters and dressings.

Attaching the ostomy pouch accessory to the ostomy pouch device is to be understood as an adhering of the adhesive element to at least one element of the ostomy pouch device, preferably the hydrocolloid plate. Preferably, attaching the ostomy pouch accessory to the ostomy pouch device via the adhesive element fixes a positioning of the ostomy pouch accessory and the ostomy pouch device relative to each other.

Preferably, the ostomy pouch accessory consists solely of the sensor element and the adhesive element. In this case, the adhesive element could, for example, be a carrier for the sensor element as well. Especially preferably, the ostomy pouch accessory comprises at least one substrate carrying the sensor element and at least one absorber, wherein the sensor element is arranged between the substrate and the absorber in a perpendicular view on a main extension plane of the substrate. The substrate may be a circuit board. The substrate carrying the sensor element is to be understood as the sensor element being irreversibly connected to the substrate. For example, the sensor element may be welded, soldered, adhered and/or riveted to the substrate. The absorber is preferably configured to absorb fluids exiting during a leakage. Advantageously, the sensor element detects fluid which has been absorbed by the absorber to determine the presence of a leakage. The sensor element may be arranged between the substrate and the absorber in its entirety in the perpendicular view. Preferably, the sensor element comprises at least one interface area protruding from between the substrate and the absorber, which especially preferably can be, preferably reversibly, coupled to a reading device. The reading device is preferably configured to process measurement data of the sensor element. Advantageously, the reading device has a set moisture threshold value, especially advantageously, exceeding this moisture threshold value causes the reading device to emit an alarm to the user. The alarm may for example be an optical alarm, an acoustic alarm and/or a vibration alarm. The reading device may be emitted by the reading device itself. Alternatively or additionally, the reading device may comprise a communication unit configured for sending a signal to an external device, such as for example a smartphone of the user or a telemedical facility, when the moisture threshold value is exceeded, causing the external device to emit the alarm. The communication unit preferably comprises at least one wireless transmitter, such as for example a radio transmitter, a Bluetooth transmitter and/or an infrared transmitter. A “main extension plane” of a body is to be understood as a plane which, among all possible planes, cuts the body in a way that creates the largest possible cross section. Preferably, the substrate and the absorber form layers in a resting state, the resting state being a state in which the substrate and the absorber are placed on a flat surface and neither bent nor folded. A body which forms a layer is to be understood as a body having a length and thickness which each are at least ten times, advantageously at least fifteen times, especially advantageously at least twenty times a thickness of the body. Preferably, the main extension plane of the substrate extends parallel to a length and width of the substrate in the resting state and preferably parallel to a main extension plane of the absorber. This way, a secure detection of fluid exiting during a leakage and entering a close vicinity of the sensor element can be achieved. Advantageously, the absorber may also be configured to hold back and/or filter the fluid until the leakage is fixed or the user is able to leave any public areas.

In a preferred embodiment of the ostomy pouch accessory, the sensor element is a printed sensor and the substrate exhibits at least one, preferably two, especially preferably all of the following properties:
1) an elongation at break of ≥300% and ≤800%
2) a tension at break of ≥30 MPa and ≤100 MPa
3) an inclusion of a polymer selected from a group consisting of a polyester, a polycarbonate, a co-polycarbonate, a polyurethane, a thermoplastic polyurethane, an acrylonitrile-butadiene-styrene copolymer, a polyethylene terephthalate, a polyamine, a polyamide, a polystyrene and a polysiloxane or a blend or mixture of at least two of them.

Preferably, the sensor element consists of an anorganic or organic dye, which is preferably a solution, dispersion or suspension of conductive micro particles and/or nano particles before it is printed. The micro particles and/or nano particles may for example comprise polyaniline, silver, gold and/or carbon or combinations of at least two of these. For example, the sensor element may be printed onto the substrate via a 2D printing process such as screen printing or inkjet printing. The sensor element may also be printed onto the substrate using a 3D printing process such as stereolithography, 3D screen printing or polyjet modeling and preferably have a predefined three dimensional shape. The given values of tension at break and elongation at break are based on the norm DIN EN ISO 527-1:2016-06. The substrate may consist entirely of one of the mentioned polymers, alternatively, the substrate may consist of a mixture of at least two of the polymers. Preferably, the substrate comprises or consists of a mixture of thermoplastic polyurethane and an acrylonitrile-butadiene-styrene copolymer, wherein a weight percentage of the acrylonitrile-butadiene-styrene copolymer preferably is 5-40 wt %, based on a total weight of the substrate. Alternatively, the substrate may comprise or consist of a mixture of a thermoplastic polyurethane and a polystyrene, wherein a weight percentage of the polystyrene preferably is 5-40 wt %, based on the total weight of the substrate. Advantageously, the substrate can be adjusted in terms of its extension at least in a partial area which is not occupied by the sensor element. Preferably, the extension of the substrate may be adjusted via at least one cutting process and/or at least one die-cutting process. This way, a scalable design of the ostomy pouch accessory may be achieved in a flexible and easy manner. Advantageously, the adjustment of the ostomy pouch accessory, for example to fit a new type of ostomy pouch device the ostomy pouch accessory is to be used with, can be performed in a simple and cost-effective manner. Especially advantageously, the ostomy pouch accessory can adapt to a shape of the skin area of the user due to the flexibility of the substrate, which allows for a secure detection of leakages regardless of the place of application and the type of user.

In a preferred embodiment of the ostomy ouch accessory, the absorber exhibits at least one, preferably all of the following properties:
1) a maximum quantity of fluid that can be absorbed of ≥40 ml
2) a pore size of ≤120 μm.

Preferably, the absorber is a super absorber and comprises, for example, a hydrogel. Especially preferably, the absorber comprises at least one fleece and/or foam. Further preferably, the absorber comprises at least one cellulose. Preferably, the absorber may comprise at least one additional functional material. Preferably, the functional material prevents any odors from escaping and comprises activated carbon and/or titanium dioxide. This way, a detection of the leakage and an prevention of an escaping of fluids and/or odors can be achieved, even for significant leakages.

Preferably, the absorber protrudes past the substrate in the perpendicular view. Preferably, the absorber comprises an additional partial area which is arranged behind the sensor element in relation to the ostomy pouch device to absorb additional liquid even after the leakage has been detected. Preferably, the additional partial area is configured to be attached to the adhesive element. Alternatively or additionally, the substrate may comprise vacancies which are arranged alongside the at least one trace of the sensor element in the perpendicular view and are configured to be attached to the adhesive element. This way, a fixation of the absorber and the substrate relative to each other can be achieved in an easy manner.

Especially advantageously, the substrate and the absorber are largely congruent in the perpendicular view. The absorber and the substrate being largely congruent is to be understood as areas of the absorber and the substrate, in the perpendicular view, overlapping each other by at least 90%, preferably at least 95% and especially preferably completely, the percentages relating to a total area of the substrate. Preferably, the absorber, the substrate and the sensor element are arranged in a sandwich structure. Especially preferably, in a cross-sectional view of the ostomy pouch accessory, from top to bottom, the sequence is adhesive element, substrate, sensor element and absorber. This way, protruding sections of the substrate or the absorber which may cause annoyances to the user, for example by getting caught on objects, can be eliminated.

In a preferred embodiment of the ostomy pouch accessory, the substrate forms a ring section in the perpendicular view, the ring section at least partially surrounding the ostomy pouch device in the application state. A ring section is to be understood as a coherent area bordered by an inner line, an outer line and two end lines. The inner line and outer line are preferably identical in shape, arranged concentrically to each other and connected at their end points via the end lines. The end lines are preferably straight. Preferably, in case the inner line and outer line extend over 360° of their center angle, the ring section forms a complete ring. The inner line and outer line may be parts of polygons and the ring section part of a polygonal ring, preferably, the inner line and the outer line are part of ovals, preferably circles and the ring section part of an oval ring, preferably a circular ring. An inner diameter of the ring section is preferably dependent on a size of the ostomy pouch device that is being used and preferably adjusted to fit this size. Preferably, the inner diameter of the ring section is ≤3 cm, further preferably ≤2 cm and especially preferably ≤1 cm smaller than a diameter of the hydrocolloid plate. Preferably, the inner diameter of the ring section is ≥1 cm, further preferably ≥1.5 cm, especially preferably ≥2 cm. Preferably, the inner diameter is ≤10 cm. Preferably, an outer diameter of the ring section is ≥1 cm bigger than the inner diameter of the ring section. The substrate at least partially surrounding the ostomy pouch device in the application state is to be understood as the substrate, in the application state, extending over at least a section of the circumference of the ostomy pouch device and/or at least a part of the substrate, preferably the part carrying the sensor element, wrapping around the ostomy pouch device. Especially preferably, the sensor element is arranged in a near vicinity of an outer edge of the hydrocolloid plate in the application state. Preferably, the traces of the sensor element each comprise at least one trace ring section with a diameter between the inner diameter and the outer diameter of the ring section. This way, a leakage can be detected in an easy manner, regardless of where the leakage is occurring. Preferably, the ostomy pouch accessory can be attached and detached in an easy manner. This is preferably achieved by the adhesive of the adhesive element exhibiting an adhesion force in a range of 1 N/20 mm to 30 N/20 mm on steel according to DIN EN 1464:2012-06 (90° shear strength test) using a pulling test machine according to DIN EN ISO 527-1:206-06.

Preferably, the ring section extends over 360° of its center angle in the perpendicular view and forms a complete ring. In this case, the sensor element extends only over a part of the ring section and a rest of the ring section is formed as a thin bridge configured to stabilize a shape of the ring section. Preferably, the thin bridge of the ring section is stretchable. This way, wrapping the ostomy pouch accessory around the ostomy pouch device is made easier.

Especially preferably, the ring section extends over at most 330°, advantageously at most 300°, especially advantageously at most 270° and preferably at most 240° of its center angle in the perpendicular view. This way, the ostomy pouch accessory can be attached in an easy manner, since the gap in the ring section allows for objects to pass through while attaching the ostomy pouch accessory to the ostomy pouch device. Preferably, the ring section is an open ring section. This way, the ostomy pouch accessory can be “hooked” onto the ostomy pouch device by guiding the ostomy pouch device through the gap in the ring section, thus allowing a quick attachment of the ostomy pouch accessory with just a few steps.

The ostomy pouch accessory preferably has a width of 5 to 50 cm, further preferably of 6 to 40 cm, especially preferably of 7 to 30 cm. The ostomy pouch accessory preferably has a length of 5 to 100 cm, further preferably of 6 to 70 cm, especially preferably of 10 to 50 cm. The ostomy pouch accessory may have any shape deemed suitable by one skilled in the art. Preferably, the ostomy pouch accessory has an oval or circular shape.

The substrate preferably has a width of 1 to 50 cm, further preferably of 2 to 20 cm, especially preferably of 5 to 10 cm. The substrate preferably has a length of 2 to 100 cm, further preferably of 5 to 70 cm, especially preferably of 10 to 50 cm. The substrate may have any shape deemed suitable by one skilled in the art. Preferably, the substrate has an oval or circular shape.

The absorber preferably has a width of 1 to 50 cm, further preferably of 2 to 20 cm, especially preferably of 5 to 10 cm. The absorber preferably has a length of 2 to 100 cm further preferably of 5 to 70 cm, especially preferably of 10 to 50 cm. The absorber may have any shape deemed suitable by one skilled in the art. Preferably, the absorber has an oval or circular shape.

Preferably, the substrate and the absorber are laid on top of each other first and are only fixed to each other via the adhesive element in the application state. Especially preferably, the ostomy pouch accessory comprises a fixation unit fixing the substrate to the absorber and comprising a partial area arranged between the sensor element and the absorber, the partial area being permeable for fluids. Preferably, the liquid-permeable partial area extends over ≥80%, preferably ≥90% of a maximum extension of the sensor element. The term “fixation unit” is meant to include both a group of fixation elements for fixing the substrate to the absorber as well as gaps between the fixation elements in this context. The fixation elements may be identical or different to each other. The fixation elements may be arranged in an irregular pattern, preferably, the fixation elements are arranged in a regular pattern, for example, the fixation elements may be arranged so that in the perpendicular view, they are placed following alongside the sensor element. Preferably, the fixation elements comprise at least one weld line, weld dot, clamp and/or rivet, each of which are spaced apart from the sensor element. In this case, the liquid-permeable partial area forms a gap between the fixation elements. Further preferably, the fixation unit is a coherent adhesive film and/or adhesive tape which is liquid-permeable in any partial area. This way, a movement of the substrate relative to the absorber can be prevented, even before the ostomy pouch accessory is attached to the ostomy pouch device. Preferably, the fixation unit does not inhibit the functionality of the sensor element.

Especially preferably, the fixation unit is a partially adhesive film, wherein the partial area is an area of the film that is devoid of adhesive. Preferably, the fixation unit consists of a multitude of adhesive dots and/or adhesive strips. Further preferably, the individual adhesive areas of the fixation unit may have any other shape. Preferably, the fixation unit comprises a pressure-sensitive sensitive and, either alternatively or additionally, a structural adhesive. This way, an even more secure design of the ostomy pouch accessory can be achieved and a movement of the substrate relative to the absorber can be achieved in an especially easy and efficient manner. Especially preferably, the fixation unit is applied onto the substrate or the absorber using a liner, which is removed before applying the other of the two bonding partners onto the partially adhesive film.

Preferably, the adhesive element is a double-sided adhesive and the ostomy pouch accessory comprises an additional cover configured to cover the adhesive element in the application state. Especially preferably, the tape is a self-adhesive cover which is arranged on a side of the substrate opposite to the sensor element in the application state. Preferably, the adhesive element comprises an adhesive film on a side facing the substrate in the application state, and a carrier on a side opposite to the substrate in the application state. The carrier preferably comprises at least a cellulose, plastic and/or foam. Preferably, the carrier is hydrophobic or comprises a hydrophobic coating. This way, a false detection of a leakage caused by an ingress of liquid into the carrier can be prevented. Preferably, the adhesive element extends at least over the entire ostomy pouch accessory and an edge area of the ostomy pouch device in the perpendicular view. Preferably, the adhesive element is arranged on a side of the substrate opposite to the sensor element, even outside of the application state. In this case, the ostomy pouch accessory preferably comprises a release liner covering a part of the adhesive area configured to be applied on a skin of the patient. Alternatively, the adhesive element is only arranged on the side of the substrate opposite to the sensor element as the ostomy pouch accessory is attached. This way, attaching the ostomy pouch accessory to the ostomy pouch device may be performed via a simple application and smoothening process.

Preferably, the adhesive element protrudes by at least 30% of a width of the substrate past both sides of the substrate in the perpendicular view and in the application state. The adhesive element may protrude a different amount at different places. Preferably, the adhesive element forms a ring section in the perpendicular view and, especially preferably, has the same shape as the substrate. This way, a sturdy attachment of the ostomy pouch accessory to the ostomy pouch device can be achieved. Advantageously, the adhesive element can be adhered to the hydrocolloid plate of the ostomy pouch device, the substrate of the ostomy pouch accessory and the skin of the user at the same time.

Preferably, the ostomy pouch accessory comprises exactly one sensor element. Especially preferably, the ostomy pouch accessory comprises a multitude of sensor elements arranged concentrically to each other. Preferably, the ostomy pouch accessory comprises two or three sensor elements. Especially preferably, neighboring sensor elements share one of the traces. Preferably, the sensor elements are arranged in equal radial distances on the substrate. This way, in addition to detecting a leakage, the severity of the leakage can be evaluated by determining the amount of moisture reaching the individual sensor elements. Advantageously, a redundancy can be provided, allowing for a detection of a leakage even if one of the sensor elements should have a defect.

The present technology further relates to a system with at least one ostomy pouch device and at least one ostomy pouch accessory. This way, a system with improved properties regarding a design can be provided.

The present technology further relates to a method of using an ostomy pouch accessory with an ostomy pouch device, preferably an ostomy pouch accessory as described previously. In this method, the ostomy pouch accessory is positioned around an edge of a hydrocolloid plate of the ostomy pouch device and fixed by placing an adhesive element of the ostomy pouch accessory on the hydrocolloid plate as well as on a skin surrounding the ostomy pouch device. This way, attaching and detaching of the ostomy pouch accessory can be achieved in an easy and intuitive manner.

All of the statements referring to the previously described ostomy pouch accessory and ostomy pouch device, such as for example measurements, properties, compositions and shapes are equally valid for the method for using the ostomy pouch accessory.

In a preferred embodiment of the method, an absorber and a substrate of the ostomy pouch accessory are separate to each other in a non-applied state of the ostomy pouch accessory and the substrate is only fixed to the adhesive element during the application of the adhesive element. This way, individual components of the ostomy pouch accessory may be replaced in case of defect in an easy manner. In order to prevent errors during the application of the ostomy pouch accessory, it is proposed that the absorber and the substrate are fixed to each other in a non-applied state of the ostomy pouch accessory and the substrate is fixed to the adhesive element before an application of the adhesive element on the hydrocolloid plate. This way, the ostomy pouch accessory can be applied directly after a purchase with just a few steps.

The ostomy pouch accessory and the method are not limited to the previously described applications and embodiments. Preferably, the ostomy pouch accessory and the method may comprise a number of elements, components, units or steps differing from these applications and embodiments while keeping the same functionality.

A method for manufacturing the ostomy pouch accessory preferably comprises the following steps: 1) Readying the substrate, 2) printing the sensor element onto the substrate, 3) applying and fixing the substrate to the absorber. Preferably, the method for manufacturing the ostomy pouch accessory may comprise an additional step in which the substrate and the absorber are fixed relative to each other by applying the adhesive element on the substrate. This way, an ostomy pouch accessory can be manufactured which provides increased comfort and flexibility.

Further advantages may be taken from the following description of the figures. The figures show an embodiment of the present technology. The figures, description and claims present combinations of a multitude of features. One skilled in the art is able to look at these features in isolation and combine them into other, suitable combinations.

For the sake of visibility, in cases where multiple identical objects are present in the figures, only one of these objects receives a reference sign.

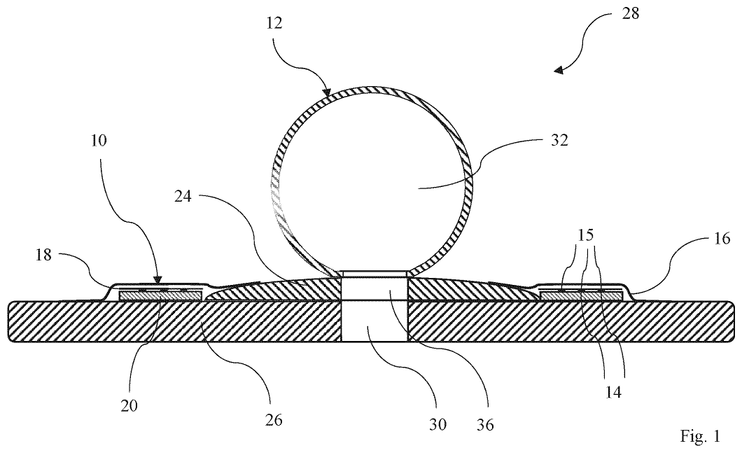

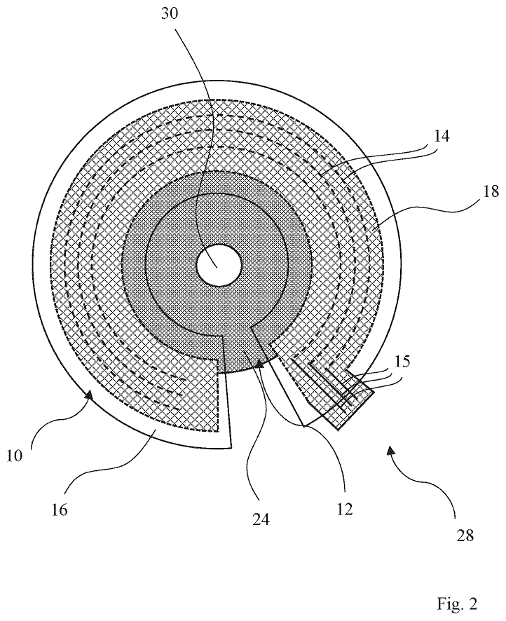

FIGS. 1 and 2 show a system 28. The system 28 is in an application state. The system 28 comprises an ostomy pouch device 12. The ostomy pouch device 12 is arranged at a stoma 30 of a user (not shown) via an opening 36. The ostomy pouch device 12 comprises a hydrocolloid plate 24. The hydrocolloid plate 24 is adhered to a skin 26 of the user and encompasses the stoma 30. The ostomy pouch device 12 comprises an ostomy pouch 32 (which is only shown in FIG. 1 for the sake of visibility). The ostomy pouch 32 is configured to take in material exiting the stoma 30 and connected to the hydrocolloid plate 24.

The system 28 comprises an ostomy pouch accessory 10. The ostomy pouch accessory 10 comprises a substrate 18. The substrate 18 is a flexible foil. The substrate 18 has an elongation at break of 500%. The substrate 18 has a tension at break of 50 MPa. The substrate 18 consists of polycarbonate. The ostomy pouch accessory 10 comprises a sensor element 14. The sensor element 14 is a printed sensor. The sensor element 14 consists of an anorganic dye comprising silver particles. The sensor element 14 is configured for a detection of a leakage of the ostomy pouch device 12. The sensor element 14 is a moisture sensor. The sensor element 14 is printed onto the substrate 18. The sensor element 14 comprises two traces. The traces are open traces devoid of turns and protrusions. Alternatively, the traces may be closed, comprise turning points and/or protrusions.

The ostomy pouch accessory 10 comprises three traces 15. The traces 15 identical to each other. Alternatively, the traces 15 may be different to each other. The ostomy pouch accessory 10 comprises an additional sensor element. The additional sensor element shares one of the traces 15 with the sensor element 14. The sensor element 14 and the additional sensor element are arranged concentrically to each other. The sensor element 14 and the additional sensor element are spaced apart in an equal radial distance.

The ostomy pouch accessory 10 comprises an absorber 20. The absorber consists of a fleece. Alternatively or additionally, the absorber 20 may comprise a foam and/or a cellulose. The absorber 20 has a maximum quantity of fluid that can be absorbed of 50 ml. The absorber 20 has a maximum pore size of 100 μm. The absorber 20 is placed on the skin 26 of the user. The substrate 18 is placed on the absorber 20. The sensor element 14 is arranged between the substrate 18 and the absorber 20. The substrate 18 and the absorber 20 are congruent to each other. The substrate 18, the sensor element 14 and the absorber 20 are arranged in a sandwich structure.

The ostomy pouch accessory 10 comprises an adhesive element 16. The adhesive element 16 provides a detachable connection between the ostomy pouch accessory 10 and the ostomy pouch device 12. The adhesive element 16 is a self-adhesive cover. The adhesive element 16 comprises a cover side and an adhesive side. The adhesive element 16 comprises an adhesive area comprising a polyurethane adhesive. The adhesive area extends over the entire adhesive side. The adhesive element 16 is arranged on a side of the substrate 18 opposite to the sensor element 14. The adhesive element 16 is applied on the substrate 18 (for the sake of visibility, in FIG. 1, there is a gap between the adhesive element 16 and the substrate). The adhesive element 16 protrudes by at least 30% of a width of the substrate 18 past both sides of the substrate 18. The adhesive element 16 is applied on an edge of the hydrocolloid place 24. The adhesive element 16 is applied on the skin 26 of the user.

The substrate 18 forms a sing section (see FIG. 2). The substrate 18 partially surrounds the ostomy pouch device 12. The ring section extends over 320° of its center angle in the perpendicular view.

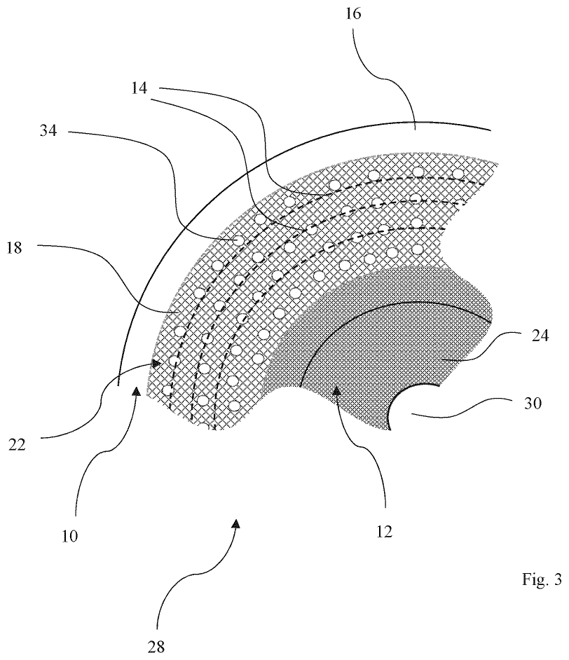

The ostomy pouch accessory 10 comprises a fixation unit 22, which is shown in FIG. 3. The fixation unit 22 fixes the substrate 18 to the absorber 20. The fixation unit 22 comprises a partial area arranged between the sensor element 14 and the absorber 20, the partial area being permeable for fluids. The fixation unit 22 is a partially adhesive film. The fixation unit 22 comprises a group of fixation elements 34 as well as gaps between the fixation elements 34. The fixation elements 34 are adhesive dots, alternatively, the fixation elements 34 could be adhesive strips. The partial area is an area of the fixation unit 22 that is devoid of adhesive.

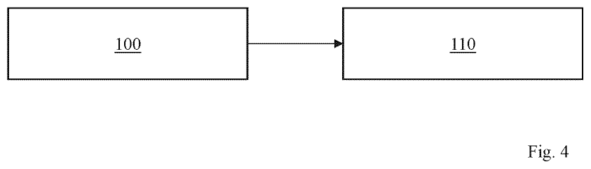

FIG. 4 shows a schematic chart for a method for using the ostomy poach accessory 10 with the ostomy pouch device 12. In a preparation step 100, the substrate 18 is fixed to the adhesive element 16 by being pressed against the adhesive area of the adhesive element 16. The preparation step 100 may be performed by the user or during a manufacturing of the ostomy pouch accessory 10.

In an application step 110, the ostomy ouch accessory 10 is positioned around an edge of the hydrocolloid plate 24 and fixed by placing the adhesive element 16 on the hydrocolloid plate 24 as well as on the skin 26. The application step 110 follows after the preparation step 100. Alternatively, the preparation step 100 may not be followed by any other step, in this case the preparation step 100 may additionally comprise an application of the substrate 18 and the absorber 20 on the skin 26 before the adhesive element 22 is placed.

1. Ostomy pouch accessory for usage with at least one ostomy pouch device, comprising at least one sensor element for a detection of a leakage of the ostomy pouch device in at least one application state, also comprising at least one adhesive element for a detachable fixation of the ostomy pouch accessory in the application state.

2. Ostomy pouch accessory according to claim 1, characterized by at least one substrate carrying the sensor element and by at least one absorber, wherein the sensor element is arranged between the substrate and the absorber in a perpendicular view on a main extension plane of the substrate.

3. Ostomy pouch accessory according to claim 2, characterized in that the sensor element being a printed sensor and the substrate exhibiting at least one, preferably two, especially preferably all of the following properties:
1) an elongation at break of ≥300% and ≤800%
2) a tension at break of ≥30 MPa and ≤100 MPa
3) an inclusion of a polymer selected from a group consisting of a polyester, a polycarbonate, a co-polycarbonate, a polyurethane, a thermoplastic polyurethane, an acrylonitrile-butadiene-styrene copolymer, a polyethylene terephthalate, a polyamine, a polyamide, a polystyrene and a polysiloxane or a blend or mixture of at least two of them.

4. Ostomy pouch accessory according to claim 2 or 3, characterized in that the absorber exhibits at least one, preferably all of the following properties:
1) a maximum quantity of fluid that can be absorbed of ≥40 ml
2) a pore size of ≤120 μm.

5. Ostomy pouch accessory according to any of the claims 2 to 4, characterized in that the substrate and the absorber are largely congruent in a perpendicular view.

6. Ostomy pouch accessory according to any of the claims 2 to 5, characterized in that the substrate forming a ring section in the perpendicular view, the ring section at least partially surrounding the ostomy pouch device in the application state.

7. Ostomy pouch accessory according to claim 6, characterized in that the ring section extends over at most 330° of its center angle in the perpendicular view.

8. Ostomy pouch accessory according to any of the claims 2 to 7, characterized by a fixation unit fixing the substrate to the absorber and comprising a partial area arranged between the sensor element and the absorber, the partial area being permeable for fluids.

9. Ostomy pouch accessory according to claim 8, characterized in that the fixation unit is a partially adhesive film, wherein the partial area is an area of the film that is devoid of adhesive.

10. Ostomy pouch accessory according to any of the claims 2 to 9, characterized in that the adhesive element is a self-adhesive cover which is arranged on a side of the substrate opposite to the sensor element in the application state.

11. Ostomy pouch accessory according to any of the claims 2 to 10, characterized in that the adhesive element protrudes by at least 30% of a width of the substrate past both sides of the substrate in the perpendicular view and in the application state.

12. Ostomy pouch accessory according to any of the preceding claims, characterized by a multitude of sensor elements arranged concentrically to each other.

13. System with at least one ostomy pouch device and at least one ostomy pouch accessory according to any of the preceding claims.

14. Method of using an ostomy pouch accessory, preferably according to one of the claims 1 to 12, with an ostomy pouch device, characterized in that the ostomy pouch accessory is positioned around an edge of a hydrocolloid plate of the ostomy pouch device and fixed by placing an adhesive element of the ostomy pouch accessory on the hydrocolloid plate as well as on a skin surrounding the ostomy pouch device.

15. Method according to claim 14, characterized in that an absorber and a substrate of the ostomy pouch accessory are fixed to each other in a non-applied state of the ostomy pouch accessory and in that the substrate is fixed to the adhesive element before an application of the adhesive element on the hydrocolloid plate.
